(19) United States
(12) Patent Application Publication
(10) Pub. No.: US 2016/0051674 A1
(43) Pub. Date: Feb. 25, 2016

(54) METHODS AND PHARMACEUTICAL COMPOSITIONS (CTPS 1 INHIBITORS, E.G. NORLEUCINE) FOR INHIBITING T CELL PROLIFERATION IN A SUBJECT IN NEED THEREOF

(71) Applicants: INSERM (INSTITUT NATIONAL DE LA SANTÉ ET DE LA RECHERCHE MÉDICALE), Faris (FR); FONDATION IMAGINE, Paris (FR); UNIVERSITÉ PARIS DESCARTES, Paris (FR); ASSISTANCE PUBLIQUE-HÔPITAUX DE PARIS (APHP), Paris (FR); THE UNIVERSTY OF MANCHESTER, Manchester (GB)

(72) Inventors: Sylvain LATOUR, Paris (FR); Alain FISCHER, Paris (FR); Emmanuel MARTIN, Paris (FR); Peter ARKWRIGHT, Manchester (GB)

(21) Appl. No.: 14/781,353

(22) PCT Filed: Apr. 17, 2014
(86) PCT No.: PCT/EP2014/057895

§ 371 Date: Sep. 30, 2015

(30) Foreign Application Priority Data
Apr. 18, 2013 (EP) 13305504.6

(51) Int. Cl. A61K 39/395; A61K 31/7068; A61K 31/713; A61K 45/06; A61K 31/655; A61K 31/436; A61K 31/185; A61K 31/353; A61K 31/404; A61K 31/215; A61K 31/4015; A61K 31/5377; A61K 31/52; A61K 38/13; G01N 33/50; A61K 31/42

(52) U.S. Cl. CPC A61K 39/3955; A61K 31/42; A61K 31/7068; A61K 31/713; A61K 45/06; A61K 31/655; A61K 31/436; A61K 31/185; A61K 31/353; A61K 31/404; A61K 31/215; A61K 31/4015; A61K 31/5377; A61K 31/52; A61K 38/13; G01N 33/505; G01N 33/5023

(57) ABSTRACT

The present invention relates to methods and pharmaceutical compositions for inhibiting lymphocyte proliferationin a subject in need thereof. In particular, the invention relates to a CTP synthase 1 (CTPS1) inhibitor for use in a method for inhibiting lymphocyte proliferationin a subject in need thereof. The invention also relates to a method for screening a plurality of test substances useful for inhibiting lymphocyte proliferationin a subject in need thereof comprising the steps consisting of i) testing each of the test substances for its ability to inhibit CTPS1 activity or expression and ii) identifying the test substance which inhibits CTPS1 activity or expression thereby to identify a test substance useful for inhibiting lymphocyte proliferationin a subject in need thereof.

### FIELD OF THE INVENTION

The present invention relates to methods and pharmaceutical compositions for inhibiting lymphocyte proliferation in a subject in need thereof.

### BACKGROUND OF THE INVENTION

Lymphocyte proliferation is the normal component of the immune reaction toward an antigen (e.g. a pathogen antigen). However in certain circumstances lymphocyte proliferation appears deleterious. For example, organ transplantation elicits a complex series of immunologic processes that are generally categorized as inflammation, immunity, tissue repair and structural reinforcement of damaged tissues. Typically T cell proliferation leads to inflammation by the secretion of proinflammatory cytokines, e.g., interleukin-2 (IL-2) and IFN-g. Accordingly, the skilled man in the art has tried to develop immunosuppressive agents. Immunosuppressive drugs fall into five groups: (i) regulators of gene expression; (ii) alkylating agents; (iii) inhibitors of de novo purine synthesis; (iv) inhibitors of de novo pyrimidine synthesis; and (v) inhibitors of kinases and phosphatases. For example, glucocorticoids exert immunosuppressive and anti-inflammatory activity mainly by inhibiting the expression of the genes for IL-2 and other mediators. Methotrexate and its polyglutamate derivatives suppress inflammatory responses through release of adenosine. Mycophenolic acid and mizoribine inhibit inosine monophosphate dehydrogenase. Mycophenolic acid induces apoptosis of activated T-lymphocytes. Cyclosporine and FK-506/Tacrolimus inhibit the phosphatase activity of calcineurin. Rapamycin inhibits signal transduction from the IL-2, epidermal growth factor and other cytokine receptors. Immunosuppressive and anti-inflammatory compounds in development include inhibitors of p38 kinase and of the type IV isoform of cyclic AMP phosphodiesterase, which is expressed in T cells. However, immunosuppressive agents are associated with toxicity due to their nonspecific immunosuppressive effects. Reducing immunosuppression can prevent side effects related to over-immunosuppression. However, since the intrinsic immunosuppressive requirements for each donor recipient pair are unknown, immunosuppressive minimization carries a potential risk of under-immunosuppression and consequent acute rejection, premature graft loss and death. A promising future application of immunosuppressive drugs is to search for agents that inhibit lymphocyte proliferation by novel mechanisms, as the currently used agents, which all possess non-specific broad immunosuppressive effects.

### SUMMARY OF THE INVENTION

The present invention relates to methods and pharmaceutical compositions for inhibiting lymphocyte proliferation in a subject in need thereof.

### DETAILED DESCRIPTION OF THE INVENTION

Lymphocyte functions triggered by antigen recognition and cosignals imply rapid and intense cell division, hence metabolism adaptation¹. The cytidine nucleotide triphosphate (CTP) is a precursor required for the metabolism of DNA, RNA and phospholipids²⁻⁴. CTP originates from two sources: a salvage pathway and a de novo synthesis pathway that depends on two enzymes, the CTP synthase (or synthetase) 1 and 2 (CTPS1 and CTPS2), although their respective roles are not known⁵⁻⁷. CTP synthase activity is a potentially important step for DNA synthesis in lymphocytes^{8,9}. Here, the inventors report the identification of a loss of function mutation (rs145092287) in CTPS1 in humans causing a novel and life threatening immunodeficiency characterized by an impaired capacity of activated T and B cells to proliferate. Proliferation in response to antigen receptor-mediated activation is defective in CTPS1-deficient subject T and B cells or in normal T cells knocked-down with shRNA for CTPS1. In contrast, proximal and distal TCR signaling events and responses were only weakly affected by the absence of CTPS1. Normal T-cell proliferation was restored in CTPS1-deficient cells by expressing wild-type CTPS1 or by addition of exogenous CTP or its nucleoside precursor, cytidine. CTPS1 expression was found to be low in resting T cells, but rapidly upregulated following TCR activation. These results highlight a key and specific role of CTPS1 in the immune system by its capacity to sustain the proliferation of activated lymphocytes during the immune response. CTPS1 may therefore represent a therapeutic target of immunosuppressive drugs that could specifically dampen lymphocyte activation.

Accordingly a first aspect of the invention relates to a method for reducing or inhibiting lymphocyte proliferation in a subject in need thereof comprising administering the subject with a therapeutically effective amount of at least one CTP synthase 1 (CTPS1) inhibitor.

In some embodiments, the method of the present invention is suitable for inhibiting or reducing T cell proliferation.

In some embodiments, the method of the present invention is suitable for inhibiting or reducing B cell proliferation.

In some embodiments, the subject is a transplanted subject. Typically the subject may have been transplanted with a graft selected from the group consisting of heart, kidney, lung, liver, pancreas, pancreatic islets, brain tissue, stomach, large intestine, small intestine, cornea, skin, trachea, bone, bone marrow, muscle, or bladder. The method of the invention is indeed particularly suitable for preventing or suppressing an immune response associated with rejection of a donor tissue, cell, graft, or organ transplant by a recipient subject. Graft-related diseases or disorders include graft versus host disease (GVDH), such as associated with bone marrow transplantation, and immune disorders resulting from or associated with rejection of organ, tissue, or cell graft transplantation (e.g., tissue or cell allografts or xenografts), including, e.g., grafts of skin, muscle, neurons, islets, organs, parenchymal cells of the liver, etc. With regard to a donor tissue, cell, graft or solid organ transplant in a recipient subject, it is believed that CTPS1 inhibitor according to the invention may be effective in preventing acute rejection of such transplant in the recipient and/or for long-term maintenance therapy to prevent rejection of such transplant in the recipient (e.g., inhibiting rejection of insulin-producing islet cell transplant from a donor in the subject recipient suffering from diabetes). Thus the method of the invention is useful for preventing Host-Versus-Graft-Disease (HVGD) and Graft-Versus-Host-Disease (GVHD). The CTPS1 inhibitor may be administered to the subject before and/or after transplantation (e.g., at least one day before transplantation, from one to five days after transplantation, etc.). In some embodiments, the CTPS1 inhibitor may be administered to the subject on a periodic basis before and/or after transplantation.

In some embodiments, the subject suffers from an autoimmune disease. As used herein, an “autoimmune disease” is a disease or disorder arising from and directed at an individual's own tissues. Examples of autoimmune diseases include, but are not limited to Addison's Disease, Allergy, Alopecia Areata, Alzheimer's disease, Antineutrophil cytoplasmic antibodies (ANCA)-associated vasculitis, Ankylosing Spondylitis, Antiphospholipid Syndrome (Hughes Syndrome), arthritis, Asthma, Atherosclerosis, Atherosclerotic plaque, autoimmune disease (e.g., lupus, RA, MS, Graves' disease, etc.), Autoimmune Hemolytic Anemia, Autoimmune Hepatitis, Autoimmune inner ear disease, Autoimmune Lymphoproliferative syndrome, Autoimmune Myocarditis, Autoimmune Oophoritis, Autoimmune Orchitis, Azoospermia, Behcet's Disease, Berger's Disease, Bullous Pemphigoid, Cardiomyopathy, Cardiovascular disease, Celiac Sprue/Coeliac disease, Chronic Fatigue Immune Dysfunction Syndrome (CFIDS), Chronic idiopathic polyneuritis, Chronic Inflammatory Demyelinating, Polyradicalneuropathy (CIPD), Chronic relapsing polyneuropathy (Guillain-Barré syndrome), Churg-Strauss Syndrome (CSS), Cicatricial Pemphigoid, Cold Agglutinin Disease (CAD), chronic obstructive pulmonary disease (COPD), CREST syndrome, Crohn's disease, Dermatitis, Herpetiformus, Dermatomyositis, diabetes, Discoid Lupus, Eczema, Epidermolysis bullosa acquisita, Essential Mixed Cryoglobulinemia, Evan's Syndrome, Exopthalmos, Fibromyalgia, Goodpasture's Syndrome, Hashimoto's Thyroiditis, Idiopathic Pulmonary Fibrosis, Idiopathic Thrombocytopenia Purpura (ITP), IgA Nephropathy, immunoproliferative disease or disorder (e.g., psoriasis), Inflammatory bowel disease (IBD), including Crohn's disease and ulcerative colitis, Insulin Dependent Diabetes Mellitus (IDDM), Interstitial lung disease, juvenile diabetes, Juvenile Arthritis, juvenile idiopathic arthritis (JIA), Kawasaki's Disease, Lambert-Eaton Myasthenic Syndrome, Lichen Planus, lupus, Lupus Nephritis, Lymphoscytic Lypophisitis, Ménière's Disease, Miller Fish Syndrome/acute disseminated encephalomyeloradiculopathy, Mixed Connective Tissue Disease, Multiple Sclerosis (MS), muscular rheumatism, Myalgic encephalomyelitis (ME), Myasthenia Gravis, Ocular Inflammation, Pemphigus Foliaceus, Pemphigus Vulgaris, Pernicious Anaemia, Polyarteritis Nodosa, Polychondritis, Polyglandular Syndromes (Whitaker's syndrome), Polymyalgia Rheumatica, Polymyositis, Primary Agammaglobulinemia, Primary Biliary Cirrhosis/Autoimmune cholangiopathy, Psoriasis, Psoriatic arthritis, Raynaud's Phenomenon, Reiter's Syndrome/Reactive arthritis, Restenosis, Rheumatic Fever, rheumatic disease, Rheumatoid Arthritis, Sarcoidosis, Schmidt's syndrome, Scleroderma, Sjörgen's Syndrome, Stiff-Man Syndrome, Systemic Lupus Erythematosus (SLE), systemic scleroderma, Takayasu Arteritis, Temporal Arteritis/Giant Cell Arteritis, Thyroiditis, Type 1 diabetes, Type 2 diabetes, Ulcerative colitis, Uveitis, Vasculitis, Vitiligo, and Wegener's Granulomatosis.

As used herein the term “CTPS1” has its general meaning in the art and refers to the CTP synthase 1. CTPS1 is a 67-kDa protein containing a CTP synthetase domain and a glutamine amide transfer domain that metabolize the formation of CTP from UTP and glutamine (Kursula, P. et al. Structure of the synthetase domain of human CTP synthetase, a target for anticancer therapy. Acta Crystallogr Sect F Struct Biol Cryst Commun 62, 613-7 (2006).).

As used herein, the term “CTPS1 inhibitor” refers to any compound natural or not which has the ability of reducing or suppressing the activity or expression of CTPS1. Typically the CTPS1 inhibitor can act directly on the activity by binding to the protein, or can act indirectly on the activity by reducing or inhibiting the expression of the enzyme. Thus CTPS1 inhibitors encompass inhibitor of CTPS1 expression. For example, CTPS1 inhibitors also include any compound that can compete with the substrate of CTPS1 (e.g. CTP or glutamine) to the corresponding catalytic domains. Typically, said inhibitor is a small organic molecule or a biological molecule (peptides, lipid, aptamer).

In some embodiments, the CTPS1 inhibitor is any functional analogue, derivative, substitution product, isomer, or homologue of the amino acid glutamine, which retain the property of glutamine to bind CTPS1 inhibitor.

The term “glutamine analogue” is intended herein to encompass any one of the above mentioned. The preparation of glutamine analogues according to the invention are prepared by conventional methods well known to the skilled in this field, see for example the references mentioned below in the context of specific embodiments, or standard reference literature.

In some embodiments, the CTPS1 inhibitor is a norleucine derivative, such as 6-diazo-5-oxo-L-norleucine (DON). DON is a glutamine analogue that inhibits a wide range of glutamine requiring reactions although the main effect seems to be on de novo purine biosynthesis and CTP synthetase in mammalian cells (Lyons, S. D., Sant, M. E., Christopherson, R. I. (1990) J. Biol. Chem. 265, 11377-11381). It blocks proliferation and has gone through extensive clinical trials as a cancer drug (reviewed in Catane, R., Von Hoff, D. D., Glaubiger, D. L. and Muggia, F. M. (1979) Cancer Treat. Rep. 63, 1033-1038; and Ahluwalia, G. S., Grem, J. L., Hao, Z., and Cooney, D. A. (1990) Pharmacol. Ther. 46, 243-271). U.S. Pat. No. 2,965,634 relates to norleucine derivatives, such as DON, and a process for the production thereof.

In some embodiments, the CTPS1 inhibitor is acivicin. Acivicin has been described in U.S. Pat. No. 5,489,562.

In some embodiments, the CTPS1 inhibitor is an analogue of UTP. Example of such an analogue is deazuridine (CAS Number 23205-42-7).

Other examples include Cyclopentenyl cytosine (CPEC), Gemcitabine (2′,2′-difluorodeoxycytidine, dFdC), actinomycin D, cycloheximide, dibutyryl cyclic AMP, and 6-azauridine.

An “inhibitor of expression” refers to a natural or synthetic compound that has a biological effect to inhibit the expression of a gene.

In some embodiments, said inhibitor of gene expression is a siRNA, an antisense oligonucleotide or a ribozyme.

Inhibitors of gene expression for use in the present invention may be based on antisense oligonucleotide constructs. Anti-sense oligonucleotides, including anti-sense RNA molecules and anti-sense DNA molecules, would act to directly block the translation of the targeted mRNA by binding thereto and thus preventing protein translation or increasing mRNA degradation, thus decreasing the level of the targeted protein (i.e. CTPS1), and thus activity, in a cell. For example, antisense oligonucleotides of at least about 15 bases and complementary to unique regions of the mRNA transcript sequence encoding the target protein can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression for use in the present invention. Gene expression can be reduced by contacting the tumor, subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of the targeted mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a particular type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Particular viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N. J., 1991).

Particular viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Typically the CTPS1 inhibitor of the invention is administered to the subject in a therapeutically effective amount.

By a “therapeutically effective amount” of the CTPS1 inhibitor of the invention as above described is meant a sufficient amount of the compound. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific CTPS1 inhibitor employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The CTPS1 inhibitor of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The CTPS1 inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifusoluble agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the particular methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

The CTPS1 inhibitor of the invention may be used in combination with any immunosuppressant well known in the art. Immunosuppressants include, but are not limited to, statins; mTOR inhibitors, such as rapamycin or a rapamycin analog; TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide. In some embodiments, the CTPS1 inhibitor of the invention may also be used in combination with anti-CD28 antibodies, IL2 antagonist or IL15 antagonists.

A further aspect of the invention relates to a method for screening a plurality of test substances useful for inhibiting lymphocyte proliferation in a subject in need thereof comprising the steps consisting of i) testing each of the test substances for its ability to inhibit CTPS1 activity or expression and ii) identifying the test substance which inhibits CTPS1 activity or expression thereby to select a test substance useful for inhibiting lymphocyte proliferation in a subject in need thereof.

Any assay well known in the art may be used for testing the ability of test substance to inhibit CTPS1 activity. In particular the assay may consist in the use of labeled substrate of the enzyme and then in determining the amount of the product of the conversation. It is merely required that the substrate is appropriately labelled so that its conversion can be detected by detecting the label in a product of the biosynthetic pathway. The substrate is typically a labelled glutamine or UTP. Typically, the labelled substrate may be non-radioactive or radioactive. For example, in case of a non-radioactive substrate, C¹³ labelled or deuterium-labelled substrates may be. For example, in case of radioactive substrates, C¹⁴-labelled or tritium-labelled substrates are particular. Typically, the labeled substrates may be added as aqueous solution with CTPS1. The concentration of the substrates in the aqueous solution may be 1 μM to 1 mM. In case of C¹⁴-labelled substrates the radioactivity is typically at least 0.1 μCi and in case of 3H-labelled substrates typically at least 1 μCi. The labelling with 14-carbon or 13-carbon may be single whereby any one of the C-positions may be labelled. Alternatively, the substrates may be multiply labelled, such as dual, triple, quadruple or quintuple. The total C-labelling is particularly particular in case of 13-carbon labelling. The labelling with deuterium or tritium may be single or multiple. Typically, the labelled substrates may be prepared enzymatically or chemically. The substrate, the test substance and the enzyme are typically incubated in time sufficient for allowing the enzymatic conversion. It is then possible to separate from the solution the CTP produced by the conservation of the substrate, by HPLC, thin layer chromatography or the like. In case of radioactive labelling, the determination of labelled product may be effected by a scintillation counter, by a phosphorimager, by a radio thin layer counter or by a radio detector in combination with a chromatographic column. Typically, a connection of the HPLC to a Flow Scintillation Analyzer (Radiomatic 150 TR, Packard) made it possible to check the radioactivity in the chromatographic peaks. For radioactivity measurements, the whole sample was usually loaded onto the column. The labeled products were quantified by measuring the peak heights and comparing them to a standard curve. In case of non-radioactive labelling, the determination may be effected conventionally by NMR spectroscopy (e.g. ¹³C-NMR) or mass spectroscopy (e.g. HPLC-MS or GC-MS). A test substance is considered as a CTPS1 inhibitor when the amount of the labeled product is lower than the amount of the labeled product determined in the absence of the test substance.

In some embodiments, the enzymatic conversion is explored in an in vitro assay, where by cells expressing the studied enzyme are used. In this particular embodiment, the screening method comprises the following steps:

a) preparing a suspension of cells expressing CTPS1 in a culture medium for supporting the metabolism of said cells

b) adding to said suspension a predetermined amount of a labeled substrate,

c) incubating the mixture obtained in step b) for a predetermined period of time at a predetermined temperature

d) separating from said incubated mixture obtained in step a fraction comprising the labelled product typically by lysing the cells to release their cellular contents,

e) detecting the concentration of the labelled product in said fraction obtained in step d),

f) repeating step b), c) d) and e) with the addition of a predetermined amount of the test substance under otherwise identical conditions,

g) determining the presence of inhibition of CTPS1 by observation of whether the concentration of labeled product detected in step f) is lower than the concentration of labeled product detected in step e).

Basically the concentrations of labeled product detected in step e) or f) represent the pool of CTP. A decrease of the CTP pool when the cells are incubated with the test substance indicates that the test substance is a CTPS1 inhibitor.

A variety of cells may be used in the in vitro assays. Typically the cell is a T cell which expresses naturally CTPS1. In some embodiments, a broad variety of host-expression vector systems may be utilized to express CTPS1 in a cell of interest. These include, but are not limited to, mammalian cell systems such as human cell lines. The mammalian cell systems may harbour recombinant expression constructs containing promoters derived from the genome of mammalian cells or from mammalian viruses (e.g., the adenovirus late promoter or the vaccine virus 7.5K promoter). DNA encoding proteins to be assayed (i.e. CTPS1) can be transiently or stably expressed in the cell lines by several methods known in the art, such as, calcium phosphate-mediated, DEAE-dextran mediated, liposomal-mediated, viral-mediated, electroporation-mediated and microinjection delivery. Each of these methods may require optimization of assorted experimental parameters depending on the DNA, cell line, and the type of assay to be subsequently employed. In addition native cell lines that naturally carry and express the nucleic acid sequences for the target protein may be used.

In well known assay in the art may also be used for determining whether a test substance is able to inhibit the expression of CTPS1. Typically, a population of cells expressing CTPS1 is cultured in the presence of the test substance and the expression level of CTPS1 is then determined and compared to the level determined in the absence of the test substance. It is concluded that the test substance is a CTPS1 inhibitor when the level of CTPS1 expression determined in the presence of the test substance is lower than the level of CTPS1 expression determined in the absence of the test substance.

The determination of the expression level of a gene can be performed by a variety of techniques. Generally, the expression level as determined is a relative expression level. More typically, the determination comprises contacting the sample with selective reagents such as probes, primers or ligands, and thereby detecting the presence, or measuring the amount, of polypeptide or nucleic acids of interest originally in the sample. Contacting may be performed in any suitable device, such as a plate, microtiter dish, test tube, well, glass, column, and so forth In some embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the sample.

In some embodiments, the expression level may be determined by determining the quantity of mRNA.

Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Typically quantitative or semi-quantitative RT-PCR is particular. Real-time quantitative or semi-quantitative RT-PCR is particularly advantageous.

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more typically 85% identical and even more typically 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin).

Probes typically comprise single-stranded nucleic acids of between 10 to 1000 nucleotides in length, for instance of between 10 and 800, more typically of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they typically hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A particular kit also includes the components necessary to determine if amplification has occurred. The kit may also include, for example, PCR buffers and enzymes; positive control sequences, reaction control primers; and instructions for amplifying and detecting the specific sequences.

In some embodiments, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Other methods for determining the expression level of said genes include the determination of the quantity of proteins encoded by said genes. Such methods comprise contacting a biological sample with a binding partner capable of selectively interacting with a marker protein present in the sample. The binding partner is generally an antibody that may be polyclonal or monoclonal, typically monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

Typically, the test substance of may be selected from the group consisting of peptides, peptidomimetics, small organic molecules, antibodies, aptamers or nucleic acids. For example the test substance according to the invention may be selected from a library of compounds previously synthesized, or a library of compounds for which the structure is determined in a database, or from a library of compounds that have been synthesized de novo. In some embodiments, the test substance may be selected form small organic molecules. As used herein, the term “small organic molecule” refers to a molecule of size comparable to those organic molecules generally sued in pharmaceuticals. The term excludes biological macromolecules (e.g.; proteins, nucleic acids, etc.); particular small organic molecules range in size up to 2000 Da, and most typically up to about 1000 Da.

The screening methods of the invention are very simple. It can be performed with a large number of test substances, serially or in parallel. The method can be readily adapted to robotics. For example, the above assays may be performed using high throughput screening techniques for identifying test substances for developing drugs that may be useful to the treatment or prevention of an inflammatory bowel disease. High throughput screening techniques may be carried out using multi-well plates (e.g., 96-, 389-, or 1536-well plates), in order to carry out multiple assays using an automated robotic system. Thus, large libraries of test substances may be assayed in a highly efficient manner. A particular strategy for identifying test substances starts with cultured cells transfected with a reporter gene fused to the promoter of any gene that is activated by the stress response pathway. More particularly, stably-transfected cells growing in wells of micro-titer plates (96 well or 384 well) can be adapted to high through-put screening of libraries of compounds. Compounds in the library will be applied one at a time in an automated fashion to the wells of the microtitre dishes containing the transgenic cells described above. Once the test substances which activate one of the target genes are identified, it is preferable to then determine their site of action in the Integrated Stress Response pathway. It is particularly useful to define the site of action for the development of more refined assays for in order to optimize the target substance.

In some embodiments, the test substances that have been positively selected may be subjected to further selection steps in view of further assaying its properties in vitro assays or in an animal model organism, such as a rodent animal model system, for the desired therapeutic activity prior to use in humans.

For example, in vitro assays may include use of B cell lines or T cell lines such as Jurkat cell line, or MOLT-4 cell line. In a particular, the method may further comprise the steps consisting of providing a B or T cell line, bringing into contact the cell line with the selected test substance, determining the proliferation level of the B or T cell line, comparing said proliferation level with the proliferation level determined in the absence of the test substance, and positively selecting the test substance when the proliferation level determined in the presence of the test substance is lower that the proliferation level determined in the absence of the test substance.

For example, assays which can be used to determine whether administration of a selected CTPS1 inhibitor is indicated, include cell culture assays in which a subject tissue sample is grown in culture, and exposed to or otherwise contacted with a the CTPS1 inhibitor, and the effect of such composition upon the tissue sample is observed. The tissue sample can be obtained by biopsy from the subject. This test allows the identification of the therapeutically most effective CTPS1 inhibitor. In various specific embodiments, in vitro assays can be carried out with representative cells of cell types involved in an autoimmune (e.g., T cells), to determine if a test substance has a desired effect upon such cell types.

Any well known animal model may be used for exploring the in vivo therapeutic effects of the screened CTPS1 inhibitors. For example, the therapeutic activity of the screened CTPS1 inhibitors can be determined by using various experimental animal models of inflammatory arthritis known in the art and described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993). Experimental and spontaneous animal models of inflammatory arthritis and autoimmune rheumatic diseases can also be used to assess the anti-inflammatory activity of the screened CTPS1 inhibitor. The effect of CTPS1 inhibitors to reduce one or more symptoms of an autoimmune disease can be monitored/assessed using standard techniques known to one of skill in the art. Peripheral blood lymphocytes counts in a mammal can be determined by, e.g., obtaining a sample of peripheral blood from said mammal, separating the lymphocytes from other components of peripheral blood such as plasma using, e.g., Ficoll-Hypaque (Pharmacia) gradient centrifugation, and counting the lymphocytes using trypan blue. Peripheral blood T cell counts in mammal can be determined by, e.g., separating the lymphocytes from other components of peripheral blood such as plasma using, e.g., a use of Ficoll-Hypaque (Pharmacia) gradient centrifugation, labeling the T cells with an antibody directed to a T cell antigen such as CD2, CD3, CD4, and CD8 which is conjugated to FITC or phycoerythrin, and measuring the number of T cells by FACS. Further, the effect on a particular subset of T cells (e.g., CD2+, CD4+, CD8+, CD4+RO+, CD8+RO+, CD4+RA+, or CD8+RA+) cells can be determined using standard techniques known to one of skill in the art such as FACS. Thus the lymphocyte proliferation in the animal model may be easily assessed. Other examples of animal models that can be used for the in vivo screening include animal for encephalomyelitis EAE, or lpr mice.

The invention will be further illustrated by the following figures and examples. However, these examples and figures should not be interpreted in any way as limiting the scope of the present invention.

### FIGURES

FIG. 1. CTPS1 is required for proliferation of T-cells in response to TCR-CD3 activation. a, Proliferation of T-cells in which CTPS1 expression was silenced with vectors containing shRNA for CTPS1 (Sh CTPS1#1 or Sh CTPS1#2) or containing a scramble shRNA (Sh scramble) with GFP gene reporter. Representative dot plots of GFP⁺ cells corresponding to transduced cells (left upper panels). Representative histograms of violet dye dilution showing the cell divisions after stimulation (left lower panels). Curves showing the percentage of GFP⁺ transduced cells in long-term expansions after repeated stimulation (middle panel). Immunoblots for CTPS1 and CTPS2 expression in transduced cells (right panels). ACTIN serves as loading control. One representative of two experiments. b,c, Proliferation of control (Ctr.) and CTPS1-deficient T-cells (patient P1.2) transduced by empty or wild-type CTPS1-containing vector. Representative histograms of violet dye dilution (b, left panels) and indexes of cell division after stimulation (b, right panels). Mean values with s.d. of triplicate in one representative of two experiments. Curves showing the percentage of GFP⁺ transduced cells same as in (a) (c, left panel). Representative data from one of 2 independent experiments. Immunoblots same as in (a) (c, right panels). d, Representative histograms of violet dye dilution showing cell divisions of control (Ctr.) and CTPS-1-deficient cells (patient P1.2). Cells were incubated with the indicated nucleotides or nucleosides before stimulation. Data from one of 3 independent experiments. e, Same as (d) excepted that control T-cells were incubated with deazauridine before and during stimulation. Data from one representative of 3 independent experiments. f, Concentration of CTP in cell extracts of T-cell blasts from healthy controls (Ctr.) and CTPS1-deficient cells (patient 1.2) after stimulation with anti-CD3/CD28 coated beads. Control cells were incubated or not with deazauridine before and during stimulation. Data from 3 independent experiments. g, Concentration of CTP in cell extracts of EBV B-cell lines from healthy controls (Ctr.), and CTPS1-deficient patients (Pat.) transduced or not with wild-type CTPS1-containing vector. P1.1 (squares), P1.2 (circles) and P2.1 (triangles). For controls, symbols correspond to different donor cells. Data from 2 independent experiments. Unpaired t-tests. ***P<0.001. h, Proliferation of CTPS1-deficient EBV B cell lines (P1.2 and P2.1) transduced by empty or wild-type CTPS1-containing vector. Curves showing the percentage of GFP⁺ transduced cells in culture.

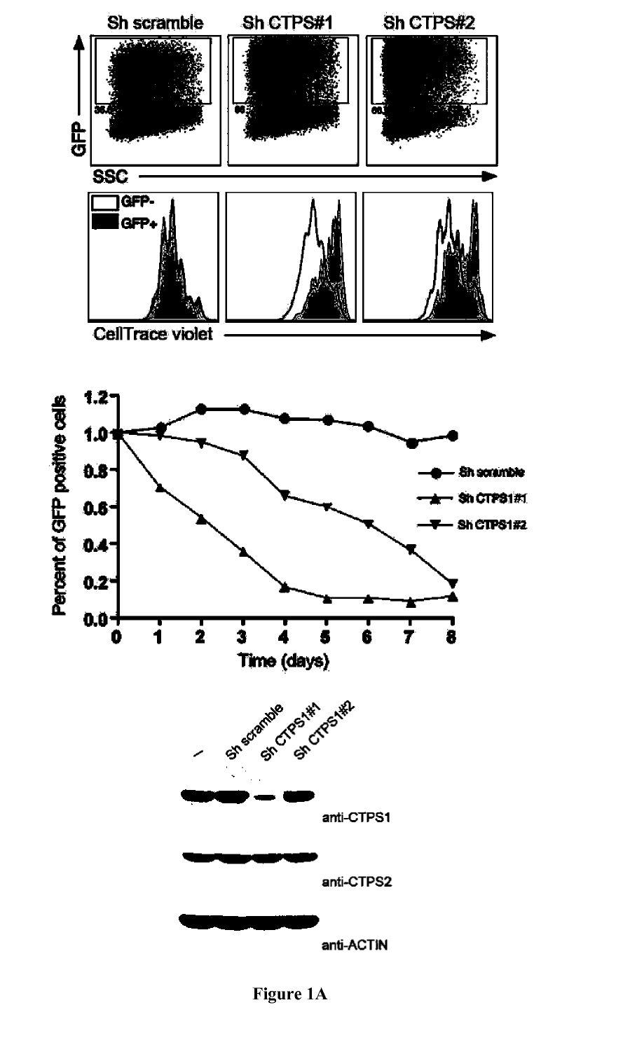

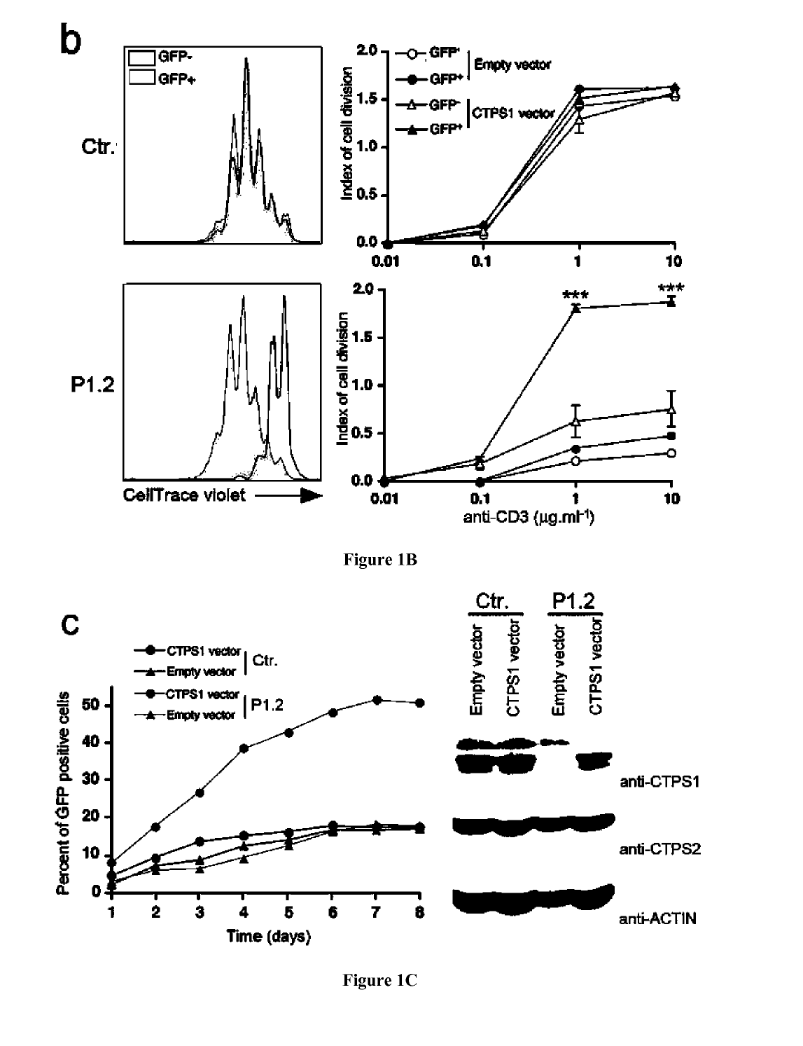

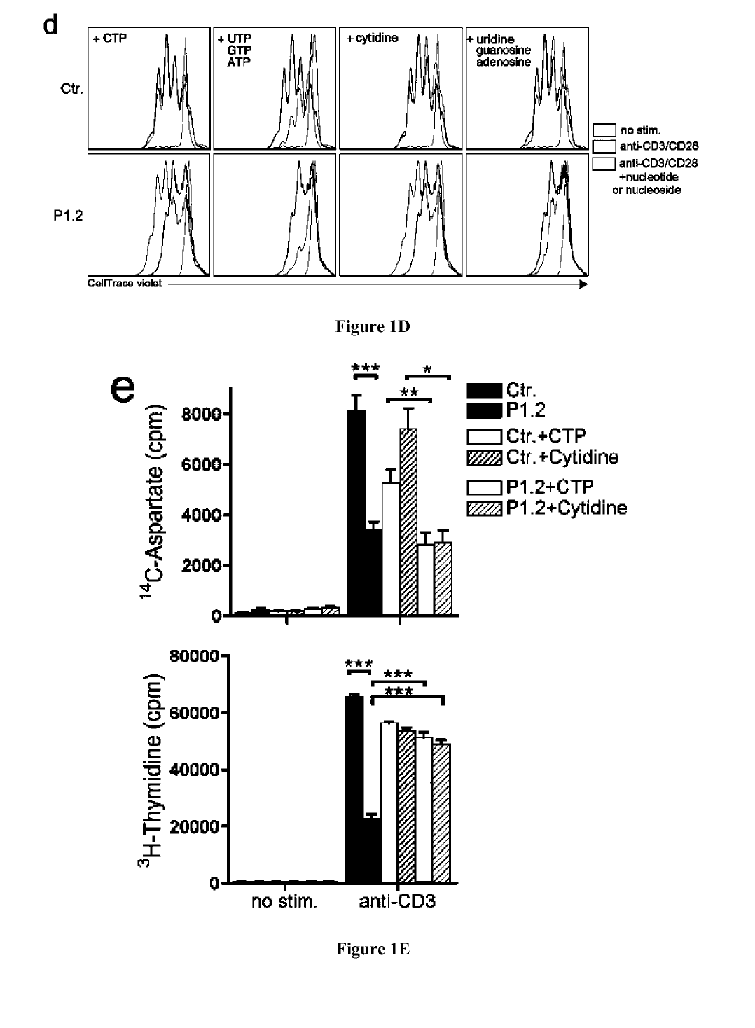

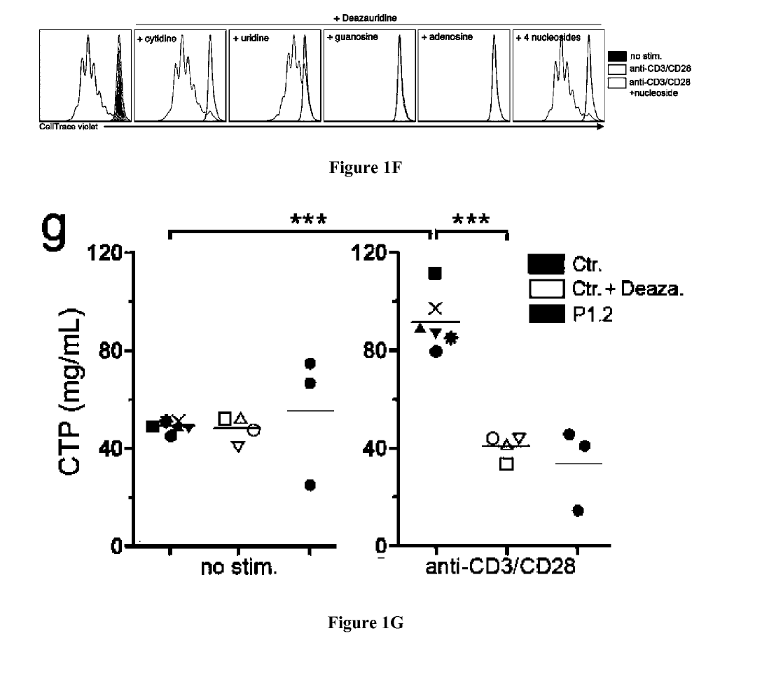

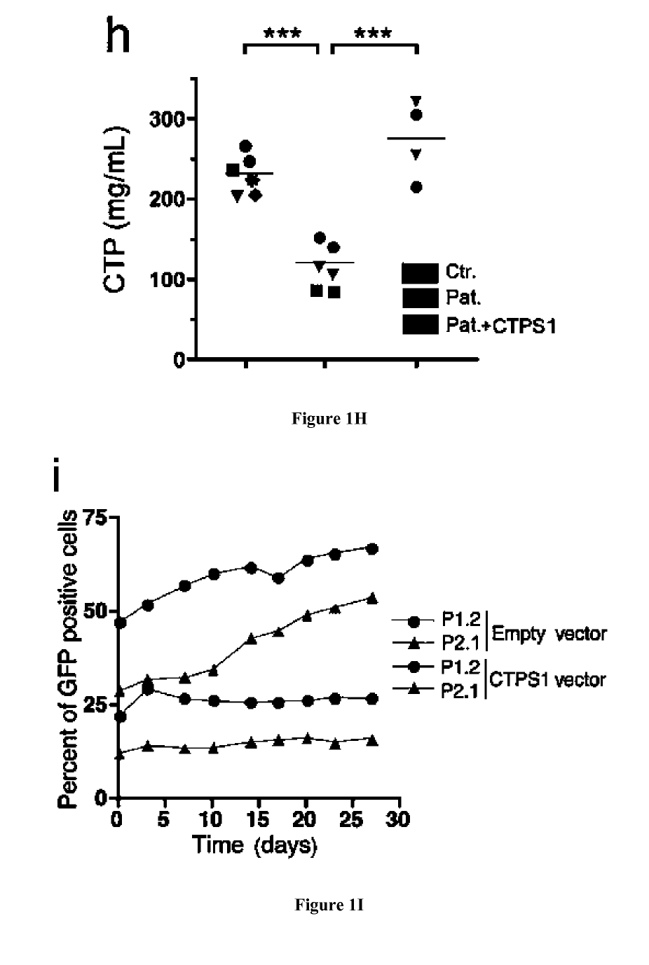

### EXAMPLE

### CTP Synthase 1 Deficiency in Humans Reveals its Central Role in Lymphocyte Proliferation

Methods:

Informed consent was obtained from donors, patients and families of patients. The study and protocols conform to the 1975 declaration of Helsinki as well as to local legislation and ethical guidelines. Genomic DNA extracted from peripheral blood cells was used for whole exome sequencing (Illumina) and sequencing of CTPS1. PBMCs were Ficoll-purified and activated with phytohemagglutinin (PHA) for 3 days, and then cultured in RPMI medium supplemented with 5% type AB human serum and IL-2 (100 UI/ml). T-cell blasts were restimulated with various mitogens and analyzed for memory and activation markers, calcium flux, cytokine secretions, apoptosis and TCR-CD3 signaling cascade molecules by immunoblotting. CTPS1 protein was detected by immunoblotting with an antibody raised against residues in the middle of the protein (341 to 355) (#SAB111071, Sigma). For proliferation and cell cycle and assays, cells were deprived of IL-2 for 3 days, incubated with the CellTrace violet dye (Invitrogen) or the 5-ethynil-2′-deoxyuridine (EdU) (Click-IT, Invitrogen) before restimulation with anti-CD3 antibodies (1 μg·ml⁻¹) alone (clone OKT3, eBiosciences) or with CD3/CD28-coated beads (Invitrogen). Proliferation was assessed after 96 hours by monitoring the dilution of CellTrace violet dye labelling. Cell cycle was determined by measuring the incorporation of EdU into newly synthesized DNA during 40 hours. The division indexes were calculated with the Flowjo software (BD Biosciences). For gene silencing of CTPS1 by shRNA expression (Openbio systems), cells were transduced at day 3 of PHA stimulation. For rescue experiments, CTPS1-deficient cells were transduced with a lentiviral vector (Invitrogen) containing the CTPS1 gene and proliferation was performed 5 days after transduction. In long-term expansions, cells were repeatedly stimulated with CD3/CD28-coated beads every 48 h and the transduced GFP⁺ cells in the cultures were determined by every 24 h. The intracellular content of nucleotides was measured by liquid chromatography-mass spectrometry (UPLC-xevoTQS, Waters). P values were calculated by two-tailed Student's t-test using PRISM software (GraphPad).

Results

We initially studied two unrelated families (family 1 and 2) originating from the northwest region of England, whose four children suffered from severe and recurrent Epstein-Barr virus (EBV) infection, in whom known primary immunodeficiencies have been excluded¹⁰ (Table 1). Four additional patients from three unrelated families (family 3 to 5) were identified thereafter out of 34 patients (33 families) tested with severe EBV infection. All patients had early onset of severe chronic viral infections, mostly caused by herpes viruses, including EBV and Varicella Zooster Virus (VZV) and, also suffered from recurrent encapsulated bacterial infections, a spectrum of infections typical of a combined deficiency of adaptive immunity (CID)¹¹ (Table 1). Overall, the clinical phenotype was severe since one patient died at 4 years of age of disseminated VZV infection and 6 patients underwent hematopoietic stem cell transplantation (HSCT) in the first years of life. Of note, none of the patients had extra-hematopoietic manifestations (Table 1).

Immunological investigations showed that patients had an inversed CD4:CD8 T-cell ratio, normal or elevated immunoglobulin levels with increased IgG in most patients but low IgG2 levels with low antibody titers to Streptococcus pneumoniae and variable lymphopenia which was exacerbated during infection episodes while other blood cell counts were usually normal. Further analyses were performed in patient P1.2 which showed naive CD4⁺ T-cell lymphopenia, increased numbers of effector memory T cells, low numbers of memory CD27⁺ B cells, a complete absence of both invariant T cell populations (CD3⁺Vα24⁺Vβ11⁺) iNKT and (CD3⁺CD161^{high}Vα7.2⁺) MAIT cells, as well as an impaired PHA- and antigen-induced proliferation of peripheral blood mononuclear cells (PBMCs).

To identify the gene defect underlying the immunodeficiency in these patients, we performed whole exome sequencing (WES) in three patients (P1.1, P1.2 and P2.1). Intersection of the genetic variations found in the three patients pointed to an unique common homozygous G to C mutation in the CTPS1 gene encoding the CTP synthase 1 at position 41475832 in chromosome 1 with an assigned rsID (rs145092287) in the dbSNP database. CTPS1 comprises 19 exons that encode a 67-kDa protein containing a CTP synthetase domain and a glutamine amide transfer domain promoting the formation of CTP from UTP and glutamine¹². The identified mutation affects a splice donor site at the junction of intron 17-18 and exon 18 (IVS18-1 G>C) leading to the expression of an abnormal transcript lacking exon 18. This splice mutation was found to be deleterious since CTPS1 protein expression could not be detected in lysates of EBV-transformed B cells and T-cell blasts from patients by using two different anti-CTPS1 antibodies. In contrast, CTPS2 was expressed normally in patient cell lysates. Four additional patients with similar clinical presentations originating from the same geographical region were also found to be homozygous for the same splice mutation (Table 1). In the five affected families, all parents were heterozygous for the mutation and tested healthy siblings were also heterozygous for the IVS18-1 G>C mutation. Sequencing of 752 healthy individuals from the northwest of England identified two heterozygous individuals for the IVS18-1 G>C mutation corresponding to an estimated frequency of homozygosity of 1:560,000. This represents more than a 10-fold increase compared to the frequency estimated from available exome databases. Homozygosity regions found by WES in P1.1, P1.2 and P2.1 and analysis polymorphic microsatellite markers in all patients revealed that they shared a same region of homozygosity of 1.1 Mb surrounding the IVS18-1 G>C mutation. All these data were indicative of a founder effect. These observations led us to conclude that the immunodeficiency resulting from the CTPS1 mutation in these patients could be primarily associated with a T-cell immunodeficiency.

We next examined CTPS1 expression in normal tissues. CTPS1 mRNA expression was comparable between the different tissues, except for T cells in which CTPS1 expression was strongly up regulated after cell activation in response to TCR-CD3 and CD28 co-stimulation. Interestingly, in lysates from T-cell blasts and T cells from PBMCs, CTPS1 protein was almost undetectable. In contrast, CTPS2 expression was readily detected. Activation of T cells by anti-CD3 antibody or phorbol 12-myristate 13-actate (PMA) and ionomycin stimulations induced CTPS1 protein expression while activation with IL-2 and/or IL-15 resulted in only weak effect. Under the same experimental conditions, CTPS2 expression was also induced but to a lesser extent. In TCR-CD3-stimulated T-cell blasts, CTPS1 protein expression was enhanced from 12 hours and persisted for up to 96 hours as a consequence of CTPS1 gene transcription activation. As expected, no expression of CTPS1 was detected in T-cell blasts from the CTPS1-deficient patient (P1.2) contrasting with detection of CTPS1 mRNA and suggesting protein instability. These data indicate that T-cell activation through the TCR results in a rapid and sustained CTPS1 protein expression. Of note, in B cells activated by anti-BCR and CpG, IL-4 and CD40L or PMA and ionomycin, CTPS1 was also found to be upregulated.

To further characterize the consequences of the CTPS1 deficiency in T cells, we investigated proximal T-cell activation signals as well as late responses. Following stimulation of T cells from patient P1.2 with anti-CD3 antibodies, CTPS1-deficient cells exhibited normal global protein tyrosine phosphorylation profile and normal phosphorylation of PKC-θ, PLCγ-1, IκBα and NFAT2c, with the exception of ERK1/2 phosphorylation which was found to be decreased. Furthermore, Ca⁺⁺ flux and late responses such as degranulation and cytokines production were found to be normal, although CD25 and CD69 upregulation were significantly decreased. We also noted that CTPS1-deficient blasts exhibited a slight but significant increase of basal and activation-induced cell death as compared to control cells. Taken together, these data suggest that CTPS1 deficiency had limited consequences in signaling downstream of TCR-CD3.

Because the pool of CTP is potentially a limiting factor for DNA synthesis^{8,13}, we carefully analyzed proliferation of CTPS1-deficient T-cells. In response to activation by antigens, anti-CD3 antibody or co-stimulation by anti-CD3 and anti-CD28 antibodies, CTPS1-deficient cells from three patients (P1.1, P1.2 and P2.2) failed to sustain proliferative responses as measured by H³-thymidine uptake and CFSE or violet cell tracer dye dilution (resulting in a weak index of cell proliferation). Uptakes of ³H-Uridine and ³H-Cytidine were also found to be impaired in activated CTPS1-deficient T cells suggesting that RNA synthesis were affected. Protein synthesis determined by ³H-Leucine uptake was also diminished when concomitantly tested. Defective proliferation of CTPS1-deficient cells was also associated with a lack of cell cycle progression since a majority of cells were arrested in the G1 phase. Because CTPS1 expression in B lymphocytes is also increased following their activation, proliferation of CTPS1-deficient B cells by anti-BCR and CpG activation was examined revealing a block in their proliferation while proliferation of IL-2-activated NK cells seemed to be less affected.

Down-regulation of CTPS1 expression in control T cells, by lentiviral transduction of two distinct shRNA together with a GFP reporter gene, led to a specific decrease in the CD3-mediated proliferation of GFP-positive cells. No changes in proliferation were detected in non-targeted GFP-negative cells or in cells targeted with a scramble shRNA. The diminished proliferation resulting from the inhibition of CTPS1 expression led to a selective cell growth disadvantage with decreased numbers of GFP targeted cells over time (middle panel). A similar decrease in proliferation rate was also observed in the Jurkat T-cell line in which CTPS1 expression was down-regulated.

Together, these results indicate that CTPS1 deficiency causes a defect in T-cell proliferation in response to TCR-CD3 activation. To formally prove the causal relationship between CTPS1 deficiency and defective T-cell proliferation, we carried out reconstitution experiments with wild-type CTPS1 or by direct addition of CTP or its cytidine precursor that acts on CTP levels via the salvage pathway. Expression of ectopic CTPS1 in CTPS1-deficient T-cells fully restored proliferation upon CD3 stimulation and enabled cells to expand selectively as shown by the accumulation of GFP-positive cells expressing CTPS1. No such effect was detected in CTPS1-deficient cells transduced with an empty vector or in control cells transduced with the CTPS1-containing vector.

Proliferation and CD25 expression of CTPS1-deficient cells also recovered to a normal level by addition of CTP or cytidine. In contrast, addition of a mix of UTP, GTP and ATP or uracil, guanine and adenosine did not result in increased proliferation of CTPS1-deficient cells. Deazauridine, an analogue of UTP and a known inhibitor of CTP synthetase activity¹⁴ completely blocked T-cell proliferation of control cells in response to CD3 activation without affecting proximal TCR-CD3-mediated responses, similar to results observed in CTPS1-deficient cells. As expected, inhibition of T-cell proliferation by deazauridine was fully reverted by addition of CTP and partially by UTP, but not by ATP or GTP. Analysis of nucleotides pools in activated CTPS1-deficient T-cell blasts and CTPS1-deficient B/EBV cell lines revealed decreased levels of CTP as also observed in activated normal cells treated with deazauridine. Defective CTPS1 expression or addition of deazauridine also led to reduced pools of ATP, GTP and UTP in activated T cells suggesting interconnection in the nucleotide pools¹⁵. In contrast, CTP levels as well as ATP, GTP and UTP were found to be normal or increased in resting CTPS1-deficient T cells as the salvage pathway is predominant in quiescent cells¹⁶. Expression of wild-type CTPS1 in CTPS1-deficient B/EBV cell lines restored levels of CTP comparable to control cells and conferred to cells a selective cell growth advantage in culture.

This study reveals a critical role for CTPS1 in promoting the proliferation of human T cells following their activation. However, proliferation of B cells was also found to be dependent of CTPS1. This may directly participate to the susceptibility to encapsulated bacterial infections seen in CTPS1-deficient patients and account for the low titers of S. pneumoniae antibodies, which is a T-independent B-cell response. The role of CTPS1 in B cells could be different or/and less important than that found in T cells. Of note, CTPS1-deficient B cells preserve an intact capacity to expand upon transformation by EBV and patients had normal Ig levels and/or elevated IgG. Decreased expansion of NK cells and low numbers of iNKT and MAIT cells might also contribute to the CTPS1 immunodeficiency as these cells have been proposed to play a role in a broad range of immune responses including anti-EBV response¹⁷⁻²⁰. The finding that CTPS1-deficiency causes no other significant clinical consequences favors a redundancy with CTPS2 activity in other cell lineages and tissues. Interestingly, pyrimidine pools including CTP have been previously shown to be strongly expanded in PHA-stimulated T cells via de novo pathways including increased CTPS activity^{8,9}. The induction of CTPS1 expression in activated T cells reported here thus appears as the major determinant of CTP pool increase. In agreement with these data, proliferation was restored to normal level by addition of CTP to CTPS1-deficient T cells. The mechanism by which TCR signaling induces a rapid expression of CTPS1 in T cells remains to be determined. It is interesting to note that T cell differentiation does not appear to be severely impaired by CTPS1 deficiency, suggesting that CTP pools in thymocytes may originate from the nucleoside salvage pathway and/or the CTPS2 activity^{8,21-23}. Notably though, CTPS1 activity is critical for the intense cell division induced by antigenic stimulation as exemplified by massive proliferation and expansion of CD8⁺ T cells during viral infections^{24,25}.

Recently, the de novo pyrimidine synthesis pathway was shown to be dependent on post-transcriptional regulation by mTORC1 and S6 protein (S6K) kinases that activate the first enzymatic steps required for pyrimidine synthesis²⁶⁻²⁸. Thus, distinct regulatory mechanisms control de novo pyrimidine synthesis. Based on the present study, CTPS1-mediated tuning of CTP synthesis in lymphocytes appears to be a key element in enabling adaptive immune responses. CTPS1-specific inhibitors would potentially be highly specific immunosuppressive drugs able to inhibit auto- or allogenic-specific T and B cell responses without additional toxicity given the lymphocyte specificity of the CTPS1-deficiency phenotype. In conclusion, our results provide the first in vivo evidence of a role of the de novo pyrimidine synthesis pathway as a critical step for proliferation of T and B lymphocytes when activated by antigens.

TABLE 1
Clinical features of patients

| | | | | | | Extra- | Outcome |
|---|---|---|---|---|---|---|---|
| | Age at 1^{st} | Viral infections | | | | hematopoietic | (age in |
| Patient | symptoms | EBV | VZV | Others | Bacterial infections | manifestations | years) |
| P1.1 | 1 yr | SIM, | no | CMV, Novovirus, Rotavirus (gut) | H. influenzae (RTI) | no | HSCT (8) |
| | | chronic viremia | | Parainfluenzae I (RTI) | | | died (8) |
| P1.2 | 1 m | SIM | no | Adenovirus, HHV-6, Novovirus (gut) | yes, n.k. (RTI) | no | alive (9) |
| P2.1 | 5 yrs | LPD (CNS) | yes | no | H. influenzae (RTI) | no | HSCT (9) |
| | | | | | | | a.w. (17) |
| P2.2 | 2 yrs | chronic viremia | no | no | S. pneumoniae, | no | HSCT (7) |
| | | | | | H. influenzae (RTI) | | a.w. (13) |
| P3.1 | 1 yr | n.k. | yes (gastritis, | no | S. pneumoniae | no | died (4) |
| | | | pneumonitis) | | (septis, meningitis) | | |
| P3.2 | 3 ms | SIM, | yes | HHV-6 | no | no | HSCT (8) |
| | | chronic viremia | | | | | a.w. (12) |
| P4 | birth | LPD (CNS) | yes | CMV, Adenovirus, Rotavirus (gut) | no | no | HSCT (6) |
| | | | | | | | died (6) |
| P5 | 3 ms | LPD (CNS, liver), | no | Novovirus (gut) | N. meningitis B | no | HSCT (1) |
| | | chronic, viremia | | Parainfluenzae III, Adenovirus, | (meningitis) | | alive (2) |
| | | | | Rhinovirus (RTI) | | | |

yr., year.
m, month.
SIM, severe infectious mononucleosis.
CNS, central nervous system.
EBV, Epstein-Barr virus.
VZV, varicella zona virus.
HHV-6, human herpes virus 6.
LPD, lymphoproliferative disease.
RTI, respiratory tract infection.
CMV, cytomegalovirus.
HSCT, hematopoietic stem cell transplantation.
n.k., not known.
a.w., alive and well.

### REFERENCES

Throughout this application, various references describe the state of the art to which this invention pertains. The disclosures of these references are hereby incorporated by reference into the present disclosure.
1. MacIver, N. J., Michalek, R. D. & Rathmell, J. C. Metabolic regulation of T lymphocytes. Annu Rev Immunol 31, 259-83 (2013).
2. Evans, D. R. & Guy, H. I. Mammalian pyrimidine biosynthesis: fresh insights into an ancient pathway. J Biol Chem 279, 33035-8 (2004).
3. Higgins, M. J., Graves, P. R. & Graves, L. M. Regulation of human cytidine triphosphate synthetase 1 by glycogen synthase kinase 3. J Biol Chem 282, 29493-503 (2007).
4. Ostrander, D. B., O'Brien, D. J., Gorman, J. A. & Carman, G. M. Effect of CTP synthetase regulation by CTP on phospholipid synthesis in Saccharomyces cerevisiae. J Biol Chem 273, 18992-9001 (1998).
5. Kassel, K. M., Au da, R., Higgins, M. J., Hines, M. & Graves, L. M. Regulation of human cytidine triphosphate synthetase 2 by phosphorylation. J Biol Chem 285, 33727-36 (2010).
6. Nadkarni, A. K. et al. Differential biochemical regulation of the URA7- and URA8-encoded CTP synthetases from Saccharomyces cerevisiae. J Biol Chem 270, 24982-8 (1995).
7. van Kuilenburg, A. B., Meinsma, R., Vreken, P., Waterham, H. R. & van Gennip, A. H. Identification of a cDNA encoding an isoform of human CTP synthetase. Biochim Biophys Acta 1492, 548-52 (2000).
8. Fairbanks, L. D., Bofill, M., Ruckemann, K. & Simmonds, H. A. Importance of ribonucleotide availability to proliferating T-lymphocytes from healthy humans. Disproportionate expansion of pyrimidine pools and contrasting effects of de novo synthesis inhibitors. J Biol Chem 270, 29682-9 (1995).
9. van den Berg, A. A. et al. Cytidine triphosphate (CTP) synthetase activity during cell cycle progression in normal and malignant T-lymphocytic cells. Eur J Cancer 31A, 108-12 (1995).
10. Wynn, R. F. et al. Treatment of Epstein-Barr-virus-associated primary CNS B cell lymphoma with allogeneic T-cell immunotherapy and stem-cell transplantation. Lancet Oncol 6, 344-6 (2005).
11. Notarangelo, L. D. Functional T cell immunodeficiencies (with T cells present). Annu Rev Immunol 31, 195-225 (2013).
12. Kursula, P. et al. Structure of the synthetase domain of human CTP synthetase, a target for anticancer therapy. Acta Crystallogr Sect F Struct Biol Cryst Commun 62, 613-7 (2006).
13. Traut, T. W. Physiological concentrations of purines and pyrimidines. Mol Cell Biochem 140, 1-22 (1994).
14. McPartland, R. P., Wang, M. C., Bloch, A. & Weinfeld, H. Cytidine 5′-triphosphate synthetase as a target for inhibition by the antitumor agent 3-deazauridine. Cancer Res 34, 3107-11 (1974).
15. Qiu, Y. et al. Mycophenolic acid-induced GTP depletion also affects ATP and pyrimidine synthesis in mitogen-stimulated primary human T-lymphocytes. Transplantation 69, 890-7 (2000).
16. van den Berg, A. A. et al. The roles of uridine-cytidine kinase and CTP synthetase in the synthesis of CTP in malignant human T-lymphocytic cells. Leukemia 8, 1375-8 (1994).
17. Le Bourhis, L., Mburu, Y. K. & Lantz, O. MAIT cells, surveyors of a new class of antigen: development and functions. Curr Opin Immunol 25, 174-80 (2013).
18. Vivier, E., Tomasello, E., Baratin, M., Walzer, T. & Ugolini, S. Functions of natural killer cells. Nat Immunol 9, 503-10 (2008).
19. Chung, B. K. et al. Innate immune control of EBV-infected B cells by invariant natural killer T cells. Blood 122, 2600-8 (2013).
20. Brennan, P. J., Brigl, M. & Brenner, M. B. Invariant natural killer T cells: an innate activation scheme linked to diverse effector functions. Nat Rev Immunol 13, 101-17 (2013).
21. Toy, G. et al. Requirement for deoxycytidine kinase in T and B lymphocyte development. Proc Natl Acad Sci USA 107, 5551-6.
22. Marijnen, Y. M. et al. Studies on the incorporation of precursors into purine and pyrimidine nucleotides via ‘de novo’ and ‘salvage’ pathways in normal lymphocytes and lymphoblastic cell-line cells. Biochim Biophys Acta 1012, 148-55 (1989).
23. Austin, W. R. et al. Nucleoside salvage pathway kinases regulate hematopoiesis by linking nucleotide metabolism with replication stress. J Exp Med 209, 2215-28 (2012).
24. Murali-Krishna, K. et al. Counting antigen-specific CD8 T cells: a reevaluation of bystander activation during viral infection. Immunity 8, 177-87 (1998).
25. Hislop, A. D., Taylor, G. S., Sauce, D. & Rickinson, A. B. Cellular responses to viral infection in humans: lessons from Epstein-Barr virus Annu Rev Immunol 25, 587-617 (2007).
26. Huang, M. & Graves, L. M. De novo synthesis of pyrimidine nucleotides; emerging interfaces with signal transduction pathways. Cell Mol Life Sci 60, 321-36 (2003).
27. Ben-Sahra, I., Howell, J. J., Asara, J. M. & Manning, B. D. Stimulation of de novo pyrimidine synthesis by growth signaling through mTOR and S6K1. Science 339, 1323-8 (2013).
28. Robitaille, A. M. et al. Quantitative phosphoproteomics reveal mTORC1 activates de novo pyrimidine synthesis. Science 339, 1320-3 (2013).

1. A method for reducing or inhibiting lymphocyte proliferation in a subject in need thereof comprising administering to the subject a therapeutically effective amount of at least one CTP synthase 1 (CTPS1) inhibitor.

2. The method of claim 1 wherein said method is used to inhibit or reduce T cell proliferation.

3. The method of claim 1 wherein said method is used to inhibit or reduce B cell proliferation.

4. The method of claim 1 wherein the subject is a transplant subject.

5. The method of claim 1 wherein the subject was transplanted with a tissue, cell, organ or graft selected from the group consisting of heart, kidney, lung, liver, pancreas, pancreatic islets, brain tissue, stomach, large intestine, small intestine, cornea, skin, trachea, bone, bone marrow, muscle, or bladder.

6. The method of claim 1 wherein said method is used to prevent or suppress an immune response associated with rejection of a donor tissue, cell, graft, or organ transplant by a recipient subject.

7. The method of claim 1 wherein said method is used to prevent acute rejection of a transplant in the recipient and/or for long-term maintenance therapy to prevent rejection of a transplant in the recipient.

8. The method of claim 1 wherein said method is used to prevent Host-Versus-Graft-Disease (HVGD) or Graft-Versus-Host-Disease (GVHD).

9. The method according to claim 1 wherein the subject suffers from an autoimmune disease or a lymphoproliferative disease or is a transplant subject.

10. The method of claim 4 wherein the CTPS1 inhibitor is administered to the subject on a periodic basis before and/or after transplantation.

11. The method of claim 1 wherein the subject suffers from an autoimmune disease.

12. The method of claim 11 wherein the autoimmune disease is selected from the group consisting of Addison's Disease, Allergy, Alopecia Areata, Alzheimer's disease, Antineutrophil cytoplasmic antibodies (ANCA)-associated vasculitis, Ankylosing Spondylitis, Antiphospholipid Syndrome (Hughes Syndrome), arthritis, Asthma, Atherosclerosis, Atherosclerotic plaque, autoimmune disease (e.g., lupus, RA, MS, Graves' disease, etc.), Autoimmune Hemolytic Anemia, Autoimmune Hepatitis, Autoimmune inner ear disease, Autoimmune Lymphoproliferative syndrome, Autoimmune Myocarditis, Autoimmune Oophoritis, Autoimmune Orchitis, Azoospermia Behcet's Disease, Berger's Disease, Bullous Pemphigoid, Cardiomyopathy, Cardiovascular disease, Celiac Sprue/Coeliac disease, Chronic Fatigue Immune Dysfunction Syndrome (CFIDS), Chronic idiopathic polyneuritis, Chronic Inflammatory Demyelinating, Polyradicalneuropathy (CIPD), Chronic relapsing polyneuropathy (Guillain-Barré syndrome), Churg-Strauss Syndrome (CSS), Cicatricial Pemphigoid, Cold Agglutinin Disease (CAD), chronic obstructive pulmonary disease (COPD), CREST syndrome, Crohn's disease, Dermatitis, Herpetiformus, Dermatomyositis, diabetes, Discoid Lupus, Eczema, Epidermolysis bullosa acquisita, Essential Mixed Cryoglobulinemia, Evan's Syndrome, Exopthalmos, Fibromyalgia, Goodpasture's Syndrome, Hashimoto's Thyroiditis, Idiopathic Pulmonary Fibrosis, Idiopathic Thrombocytopenia Purpura (ITP), IgA Nephropathy, immunoproliferative disease or disorder (e.g., psoriasis), Inflammatory bowel disease (IBD), including Crohn's disease and ulcerative colitis, Insulin Dependent Diabetes Mellitus (IDDM), Interstitial lung disease, juvenile diabetes, Juvenile Arthritis, juvenile idiopathic arthritis (JIA), Kawasaki's Disease, Lambert-Eaton Myasthenic Syndrome, Lichen Planus, lupus, Lupus Nephritis, Lymphoscytic Lypophisitis, Ménière's Disease, Miller Fish Syndrome/acute disseminated encephalomyeloradiculopathy, Mixed Connective Tissue Disease, Multiple Sclerosis (MS), muscular rheumatism, Myalgic encephalomyelitis (ME), Myasthenia Gravis, Ocular Inflammation, Pemphigus Foliaceus, Pemphigus Vulgaris, Pernicious Anaemia, Polyarteritis Nodosa, Polychondritis, Polyglandular Syndromes (Whitaker's syndrome), Polymyalgia Rheumatica, Polymyositis, Primary Agammaglobulinemia, Primary Biliary Cirrhosis/Autoimmune cholangiopathy, Psoriasis, Psoriatic arthritis, Raynaud's Phenomenon, Reiter's Syndrome/Reactive arthritis, Restenosis, Rheumatic Fever, rheumatic disease, Rheumatoid Arthritis, Sarcoidosis, Schmidt's syndrome, Scleroderma, Sjörgen's Syndrome, Stiff-Man Syndrome, Systemic Lupus Erythematosus (SLE), systemic scleroderma, Takayasu Arteritis, Temporal Arteritis/Giant Cell Arteritis, Thyroiditis, Type 1 diabetes, Type 2 diabetes, Ulcerative colitis, Uveitis, Vasculitis, Vitiligo, and Wegener's Granulomatosis.

13. The method of claim 1 wherein the CTPS1 inhibitor is any functional analogue, derivative, substitution product, isomer, or homologue of the amino acid glutamine, which retain the property of glutamine to bind CTPS1 inhibitor.

14. The method of claim 1 wherein the CTPS1 inhibitor is a norleucine derivative.

15. The method of claim 1 wherein the CTPS1 inhibitor is acivicin.

16. The method of claim 1 wherein the CTPS1 inhibitor is an analogue of UTP.

17. The method of claim 16 wherein the analogue of UTP is deazuridine.

18. The method of claim 1 wherein the CTPS1 inhibitor is selected from the group consisting of Cyclopentenyl cytosine (CPEC), Gemcitabine (2′,2′-difluorodeoxycytidine, dFdC), actinomycin D, cycloheximide, dibutyryl cyclic AMP, and 6-azauridine.

19. The method of claim 1 wherein the CTPS1 inhibitor is an inhibitor of CTPS1 expression.

20. The method of claim 19 wherein the CTPS1 inhibitor is a siRNA or an antisense oligonucleotide.

21. The method of claim 1 wherein the CTPS1 inhibitor is used in combination with at least one immunosuppressant.

22. The method of claim 21 wherein the at least one immunosuppressant is selected from the group consisting of statins; mTOR inhibitors; TGF-β signaling agents; TGF-β receptor agonists; histone deacetylase inhibitors; corticosteroids; inhibitors of mitochondrial function; NF-κβ inhibitors, Dexamethasone, TCPA-1, IKK VII; adenosine receptor agonists; prostaglandin E2 agonists (PGE2); phosphodiesterase inhibitors; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors; autophagy inhibitors; aryl hydrocarbon receptor inhibitors; proteasome inhibitor I (PSI); oxidized ATPs indoleamine 2,3-dioxygenase (IDO), vitamin D3, cyclosporins, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), COX inhibitors, niflumic acid, estriol and triptolide.

23. The method of claim 1 wherein the CTPS1 inhibitor is administered in combination with anti-CD28 antibodies, IL2 antagonist or IL15 antagonists.

24. A method for screening a plurality of test substances for the ability to inhibit lymphocyte proliferation in a subject in need thereof comprising the steps of i) testing each of the test substances for its ability to inhibit CTPS1 activity or expression and ii) identifying test substances which inhibit CTPS1 activity or expression thereby identifying test substances useful for inhibiting lymphocyte proliferation in a subject in need thereof.

25. The method of claim 24 wherein the method comprises the following steps:
a) preparing a suspension of cells expressing CTPS1 in a culture medium for supporting the metabolism of said cells
b) adding to said suspension a predetermined amount of a labeled substrate,
c) incubating the mixture obtained in step b) for a predetermined period of time at a predetermined temperature
d) separating from said incubated mixture obtained in step c) a fraction comprising the labelled product by lysing the cells to release their cellular contents,
e) detecting the concentration of the labelled product in said fraction obtained in step d),
f) repeating step b), c) d) and e) with the addition of a predetermined amount of the test substance under otherwise identical conditions,
g) determining the presence of inhibition of CTPS1 by observation of whether the concentration of labeled product detected in step f) is lower than the concentration of labeled product detected in step e).

26. The method of claim 25 further comprising the steps of providing a B or T cell line, bringing the B or T cell line into contact with the selected test substance, determining the proliferation level of the B or T cell line, comparing said proliferation level with the proliferation level determined in the absence of the test substance, and positively selecting the test substance when the proliferation level determined in the presence of the test substance is lower that the proliferation level determined in the absence of the test substance.

27. The method of claim 25 which comprises further comprising a step of testing the test substance in animal model.

28. The method of claim 14, wherein said norleucine derivative is 6-diazo-5-oxo-L-norleucine (DON)

29. The method of claim 22, wherein said mTOR inhibitors, such as rapamycin or a rapamycin analog

30. The method of claim 22, wherein said histone deacetylase inhibitors, such as Trichostatin A

31. The method of claim 22, wherein said inhibitors of mitochondrial function is rotenone.

32. The method of claim 22, wherein said NF-κβ inhibitors, such as 6Bio

33. The method of claim 22, wherein said prostaglandin E2 agonists (PGE2), such as Misoprostol

34. The method of claim 22, wherein said phosphodiesterase inhibitors, such as phosphodiesterase 4 inhibitor (PDE4), such as Rolipram
PI3 KB inhibitors, such as TGX-221;

35. The method of claim 22, wherein said autophagy inhibitors, such as 3-Methyladenine

36. The method of claim 22, wherein said oxidized ATPs, such as P2X receptor blockers

37. The method of claim 22, wherein said oxidized ATPs, such as P2X receptor blockers

38. The method of claim 22, wherein said cyclosporins, such as cyclosporine A

39. The method of claim 22, wherein said aspirin and other COX inhibitors
